(19) United States
(12) Patent Application Publication
(10) Pub. No.: US 2024/0299300 A1
(43) Pub. Date: Sep. 12, 2024

(54) LIPOSOME VITAMIN C PREPARATION

(71) Applicant: Pedro P Perez, Miami, FL (US)

(72) Inventor: Pedro P Perez, Miami, FL (US)

(21) Appl. No.: 18/595,072

(22) Filed: Mar. 4, 2024

Related U.S. Application Data
(60) Provisional application No. 63/450,347, filed on Mar. 6, 2023.

(51) Int. Cl. A61K 9/127; A61K 31/375; A61P 39/06

(52) U.S. Cl. CPC A61K 9/127; A61K 31/375; A61P 39/06

(57) ABSTRACT

The present invention relates to liposome vitamin C preparations which enhance absorption of vitamin C into cells and prolong the retention of liposome vitamin C within the blood plasma and tissue of mammals, such as humans. The liposome vitamin C preparations of the present invention include amphipathic and amphiphilic molecules which improve the absorption of liposome vitamin C resulting in higher plasma and cellular levels.

### CLAIM OF PRIORITY

The present application is based on and a claim priority is made under 35 U.S.C Section 119(e) to a provisional patent application that is currently pending in the U.S. Patent and Trademark Office, namely, that having Ser. No. 63/450,347, filed on Mar. 6, 2023, which is incorporated herein by reference.

### BACKGROUND OF THE INVENTION

### Field of the Invention

The present invention relates to liposome vitamin C preparations having enhanced bioavailability.

### Description of the Related Art

The term “vitamin C,” unless otherwise stated, refers to ascorbic acid and pharmaceutically acceptable salts thereof, including, but not limited to, mineral salts of ascorbic acid, effervescent vitamin C (e.g., a combination of ascorbic acid, citric acid and sodium bicarbonate), chelates of ascorbic acid, microencapsulated of vitamin C, liposomal of vitamin C, and alkaline salts of ascorbic acid, as would be understood by those skilled in the art.

The term “liposome,” unless otherwise stated, refers to a small vesicle, spherical in shape, having at least one lipid bilayer. Due to their hydrophobicity and/or hydrophilicity, biocompatibility, particle size and many other properties, liposomes can be used as active ingredient delivery vehicles for administration of pharmaceutical, nutraceuticals and nutrients, as would be understood by those skilled in the art.

The term “amphipathic,” unless otherwise stated, refers to molecule that is composed of a hydrophilic head and a hydrophobic tail. The hydrophilic head is the polar region of the molecule and can interact with water. The hydrophobic tail is the nonpolar region and does not interact with water, as would be understood by those skilled in the art.

The term “amphiphilic,” unless otherwise stated, refers to is a chemical compound possessing both hydrophilic (water-loving, polar) and lipophilic (fat-loving) properties, as would be understood by those skilled in the art.

The term “glycerophospholipid,” unless otherwise stated, refers to are glycerol-based phospholipids. They are the main component of biological membranes, as would be understood by those skilled in the art.

The term “phospholipid,” unless otherwise stated, refers to are a class of lipids whose molecule has a hydrophilic “head” containing a phosphate group and two hydrophobic “tails” derived from fatty acids. They are the main component of biological membranes, as would be understood by those skilled in the art.

The term “sphingophospholipid,” unless otherwise stated, refers to a class of lipids containing a backbone of sphingoid bases, a set of aliphatic amino alcohols that includes sphingosine. These compounds play important roles in signal transduction and cell recognition, as would be understood by those skilled in the art.

According to the National Institute of Health and the Food and Nutritional Board of the National Academy of Science, vitamin C is an essential nutrient involved in many biological functions. Vitamin C can only be acquired through diet (i.e., food or nutritional supplement). Vitamin C has been implicated as an important dietary component as it is required for physiological and metabolic activities including the development of healthy neurons, prevention of neurodegenerative diseases, wound healing, maintenance of a healthy immune system, excellent antioxidant and free radical scavenging capabilities, superior uptake, bioavailability and cell retention, reduction of plasma levels of inflammatory and oxidative stress markers, reduces plasma levels of C-reactive protein and oxidized LDL, more rapidly absorbed and leads to higher serum vitamin C levels.

Furthermore, vitamin C contributes to the health of the cardiovascular, immunological and nervous systems, and also supports healthy bone, lung, skin, and wound-healing activities. The mechanisms through which vitamin C works generally pertain to use as an enzyme cofactor or directly as an antioxidant. While extracellular vitamin C is important in free-radical scavenging, particularly with LDL metabolism and lung protection, most vitamin C antioxidant activity and enzyme support is intracellular. Therefore, the delivery of vitamin C across the cell membrane and into the cell compartments directly impacts how effective the vitamin C is and whether the vitamin C can arrive at the needed site for healthy function.

Given the importance of vitamin C, the bioavailability of vitamin C has been the focus of intense research. An improvement in absorption and retention of vitamin C in blood plasma or tissue would increase the beneficial effects of vitamin C. Thus, there is a continuing need for vitamin C preparations having enhanced bioavailability.

### SUMMARY OF THE INVENTION

In view of the continuing need for vitamin C preparations having enhanced bioavailability, the present invention relates to a liposome vitamin C preparation which enhances absorption of vitamin C into cells and prolongs the retention of vitamin C within the blood plasma and tissue of mammals, such as humans. Furthermore, the inclusion of an amphipathic and amphiphilic molecule component improves the absorption of vitamin C, resulting in higher plasma and cellular levels. Thus, the present invention advantageously provides for a means to enhance the bioavailability of liposome vitamin C, an essential nutrient involved in many biological functions.

In advance of the foregoing, the term “about” can be defined as what one skilled in the art would understand “about” to mean, and the term “about” includes a 5% tolerance on both lower and upper bounds, if applicable.

In more specific terms, the liposome vitamin C preparation-preferably formulated as an oral dosage form, such as a tablet, capsule, softgel, or gummy—is comprised of a homogenous high shear mixture which is further comprised of a vitamin C component and an amphipathic and amphiphilic molecule component. The vitamin C component of the homogenous high shear mixture comprises at least about 74% by weight of the total weight of the preparation. In one embodiment of the present invention, the vitamin C component comprises about 0.01 g to 2.0 g of vitamin C. The amphipathic and amphiphilic molecule components, on the other hand, comprises at least about 23% by weight of the total weight of the preparation. The amphipathic and amphiphilic molecule component further comprises at least one unsaturated ω-9 C₁₈-C₂₄ fatty acid and at least one phospholipid and at least one glycerophospholipid and at least one sphingophospholipid. By way of non-limiting example, the amphipathic and amphiphilic molecule component may further comprise a combination of at least one of the following: (i) at least one saturated straight C₁₄-C₂₄ fatty acid; (ii) at least one unsaturated ω-3 C₁₆-C₂₄ fatty acid; (iii) at least one unsaturated ω-6 C₁₈-C₂₂ fatty acid; and (iv) at least one unsaturated ω-7 C₁₆-C₂₀ fatty acid; and (v) at least one unsaturated ω-9 C₁₈-C₂₄ fatty acid; and (vi) at least one phospholipid at least one glycerophospholipid and at least one sphingophospholipid. According to one embodiment of the present invention, the homogenous high shear mixture comprises a weight ratio of vitamin C to amphipathic and amphiphilic molecules ranging from about 1000:1 to about 10:1. According to another embodiment, the weight ratio ranges from about 100:1 to about 6:1 to about 1:1 to about 1:3.

In one embodiment of the present invention, the homogenous high shear mixture may comprise a vitamin C component, an amphipathic and amphiphilic molecule component, and a bioflavonoid component. In such an embodiment, the bioflavonoid component comprises at least about 0.1% by weight of the total weight of the preparation. By way of non-limiting example, the homogenous high shear mixture may comprise at least about 74% by weight of the vitamin C component, at least about 0.1% to 65% by weight of the amphipathic and amphiphilic molecule component, and at least about 0.1% to 5% by weight of the bioflavonoid component. By way of another non-limiting example, the homogenous high shear mixture may comprise at least about 70% to 35% by weight of the vitamin C component, at least about 1% to 65% by weight of the amphipathic and amphiphilic molecule component, and at least about 0.1% to 5% by weight of the bioflavonoid component.

In another embodiment of the present invention, the homogenous high shear mixture may comprise at least about 0.01% by weight of one of at least one unsaturated ω-9 C₁₈-C₂₄ fatty acid, and at least about 0.1% by weight of one of at least one amphipathic and amphiphilic molecule component, based upon 100% total weight of the preparation. By way of non-limiting example, the homogenous high shear mixture may further comprise a combination of at least one of the following: (i) at least about 0.01% by weight of at least one saturated straight C₁₄-C₂₄ fatty acid; (ii) at least about 0.01% by weight of at least one unsaturated ω-3 C₁₆-C₂₄ fatty acid; (iii) at least about 0.01% by weight of at least one ω-6 C₁₈-C₂₂ fatty acid; (iv) and at least about 0.01% by weight of at least one ω-7 C₁₆-C₂₀ fatty acid; (v) and at least about 0.01% by weight of at least one ω-9 C₁₈-C₂₄ fatty acid; (vi) and at least about 0.1% by weight of at least one amphipathic and amphiphilic molecule component, all of the foregoing based upon 100% total weight of the preparation.

In yet another embodiment of the present invention, the amphipathic and amphiphilic molecule component may comprise:
about 0.2-10.0 units (by weight) myristic acid;
about 0.2-10.0 units (by weight) pentadecanoic acid;
about 10-70.0 units (by weight) palmitic acid;
about 0.2-10.0 units (by weight) palmitoleic acid;
about 0.2-10.0 units (by weight) margaric acid;
about 0.5-60.0 units (by weight) stearic acid;
about 0.5-50.0 units (by weight) vaccenic acid;
about 5-80.0 units (by weight) oleic acid;
about 1-200.0 units (by weight) linoleic acid;
about 5-60.0 units (by weight) alpha linolenic acid;
about 5-60.0 units (by weight) linolenic acid;
about 0.2-10.0 units (by weight) arachidic acid;
about 0.2-10.0 units (by weight) gondoic acid;
about 0.2-10.0 units (by weight) behenic acid;
about 0.2-10.0 units (by weight) lignoceric acid;
about 0.2-10.0 units (by weight) nervonic acid;
about 10-50.0 units (by weight) 2-Lysophosphatidylcholine;
about 10-50.0 units (by weight) Diphosphatidylglycerole;
about 0.2-10.0 units (by weight) Lyso-Phosphatific Acid;
about 0.2-10.0 units (by weight) 1-Lyso-Phosphatidylcholine;
about 0.2-10.0 units (by weight) Lyso-Phosphatidylethanolamine;
about 1-50.0 units (by weight) N-Acyl-Phosphatidylethanolamine;
about 1-50.0 units (by weight) Phosphatidic Acid;
about 10.0-200.0 units (by weight) Phosphatidylcholine;
about 10.0-150.0 units (by weight) Phosphatidylethanolamine;
about 1-50.0 units (by weight) Phosphatidylglycerole;
about 10.0-200.0 units (by weight) Phosphatidylinositol; and
about 1-50.0 units (by weight) Phosphatidylserine,
   all based upon 100% total weight of the amphipathic and amphiphilic molecule component in the preparation.

By way of additional non-limiting example, the homogenous high shear mixture may comprise at least about 74% by weight of the vitamin C component and at least about 0.1% by weight of the amphipathic and amphiphilic molecule component, based upon 100% total weight of the preparation. More preferably, in such an example, the homogenous high shear mixture may comprise about 35% to about 71% by weight of the vitamin C component and from about 1% to about 65% by weight of the amphipathic and amphiphilic molecule component. According to yet another embodiment, the homogenous high shear mixture may comprise about 35% to about 70% by weight of the vitamin C component and from about 2% to about 65% (e.g., about 46%) by weight of the amphipathic and amphiphilic molecule component.

According to one embodiment, the weight percentage of amphipathic and amphiphilic molecules in the amphipathic and amphiphilic molecule component preferably ranges from about 23.0% or 46.0% to 65.0% by weight, based upon the total weight of the preparation. By way of non-limiting example, the homogenous high shear mixture may comprise about 23% to about 1.8% by weight of amphipathic and amphiphilic molecules, and even more desirably, about 1.0% to about 65% by weight of amphipathic and amphiphilic molecules, based upon the total weight of the preparation. In yet another embodiment of the present invention, the amphipathic and amphiphilic molecule component is comprised of at least one of the following amphipathic and amphiphilic molecules at the following weight percentages:
about 0.02-1.0% (by weight) myristic acid;
about 0.02-1.0% (by weight) pentadecanoic acid;
about 1-7.0% (by weight) palmitic acid;
about 0.02-1.0% (by weight) palmitoleic acid;
about 0.02-1.0% (by weight) margaric acid;
about 0.05-6.0% (by weight) stearic acid;
about 0.05-5.0% (by weight) vaccenic acid;
about 0.5-8.0% (by weight) oleic acid;
about 1-20.0% (by weight) linoleic acid;
about 0.5-6.0% (by weight) alpha linolenic acid;
about 0.5-6.0% (by weight) linolenic acid;
about 0.02-1.0% (by weight) arachidic acid;
about 0.02-1.0% (by weight) gondoic acid;
about 0.02-1.0% (by weight) behenic acid;
about 0.02-1.0% (by weight) lignoceric acid;
about 0.02-1.0% (by weight) nervonic acid;
about 0.1-5.0% (by weight) 2-Lysophosphatidylcholine;
about 0.1-5.0% (by weight) Diphosphatidylglycerole;
about 0.02-1.0% (by weight) Lyso-Phosphatific Acid;
about 0.02-1.0% (by weight) 1-Lyso-Phosphatidylcholine;
about 0.02-1.0% (by weight) Lyso-Phosphatidylethanolamine;
about 0.1-5.0% (by weight) N-Acyl-Phosphatidylethanolamine;
about 0.1-5.0% (by weight) Phosphatidic Acid;
about 1.0-20.0% (by weight) Phosphatidylcholine;
about 1.0-15.0% (by weight) Phosphatidylethanolamine;
about 0.1-5.0% (by weight) Phosphatidylglycerole;
about 1.0-20.0% (by weight) Phosphatidylinositol; and
about 0.1-5.0% (by weight) Phosphatidylserine,
   all based upon 100% total weight of the amphipathic and amphiphilic molecule component in the preparation.

With regard to the amphipathic and amphiphilic molecule component of the present invention, the amphipathic and amphiphilic molecule component comprises amphipathic and amphiphilic molecules which, in one embodiment of the present invention, are extracted from natural waxes and natural oils. Suitable sources of amphipathic and amphiphilic molecules may include, but are not limited to, sugar cane wax, rice bran wax, carnauba wax, candelilla wax, Japan wax, ouricury wax, bayberry wax, shellac wax, sunflower wax, orange wax, beeswax, rice brand oil, sunflower lecithin, sunflower oil, canola oil, flax seed oil, wheat germ oil, MCT oil, and hemp oil. According to one embodiment of the present invention, the set of sources of amphipathic and amphiphilic molecules comprises rice bran wax, carnauba wax, candelilla wax, beeswax, rice brand oil, sunflower lecithin, sunflower oil, canola oil, and hemp oil. According to another embodiment of the present invention, the set of sources of amphipathic and amphiphilic molecules comprises rice bran wax, rice brand oil, sunflower lecithin and sunflower oil.

With regard to the bioflavonoid component of the present invention, the bioflavonoid component comprises bioflavonoids that may include, but are not limited to: rutin, naringin, hesperidin, neohesperidin, neohesperidin dihydrochalcone, naringenin, hersperitin, nomilin, didymin, narirutin and gallic acid.

According to one embodiment of the present invention, the bioflavonoid component comprises hesperidin, gallic acid, and optionally other bioflavonoids. According to another embodiment of the present invention, the bioflavonoid component comprises hesperidin, didymin, narirutin, and optionally other bioflavonoids. By way of non-limiting example, the bioflavonoid component may comprise:
about 1000-10000 ppm hesperidin;
about 15-500 ppm didymin;
about 30-500 ppm narirutin; per gram of bioflavonoids.
   By way of additional non-limiting example, the bioflavonoid component may comprise:
about 1000-10000 ppm hesperidin;
about 15-500 ppm didymin;
about 30-500 ppm narirutin; per gram of bioflavonoids.

In some embodiments of the present invention, the preparations and/or formulations which may comprise controlled release formulations, sustained release implants, healthy beverages, foodstuffs, dietary supplements, topical compositions, and a wide variety of food applications-themselves may comprise a combination of active ingredients that can be administered to both humans and animals.

In other embodiments of the present invention, the liposome vitamin C preparation can be administered to a human subject(s) to (i) promote a healthy nervous system; (ii) prevent or decrease the risk of developing a neurodegenerative disease; (iii) enhance NGF-mediated neurite outgrowth; (iv) promote wound healing; (v) enhance fibroblast adhesion to and the interaction with the extracellular matrix; (vi) protect the immune system from xenobiotics; (vii) decrease the risk of developing an oxidative pathogenesis; and (viii) decrease the risk of developing cancer, cardiovascular diseases, respiratory infections, pulmonary diseases, lung infections, atherosclerosis, respiratory diseases, and other age-related diseases associated with cytotoxic, genotoxic, and proinflammatory mechanisms. In such embodiments, desired results may be achieved by first recognizing the efficacy of the present invention to accomplish one of the above-referenced purposes (e.g., to promote a healthy nervous system) and second, after such recognition, attending to the oral administration of an effective dose of the present invention to the appropriate human subject.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic, side perspective view of one embodiment of the liposome vitamin C preparation invention.

FIG. 2 is a schematic, top perspective view of one embodiment of the homogenous high shear mixture.

FIG. 3 is a schematic, top perspective view of another embodiment of the homogenous high shear mixture.

FIG. 4 is a TEM (Transmission electron microscope)-negative stain images of Vitamin C (PureWay-C®) fine powder liposomes manufactured by high shear mixing by the procedure described in Example 1 and as measured by the procedure described in Example 2.

FIG. 5 is a Cryo-TEM (Transmission electron microscope) images of Vitamin C (PureWay-C®) fine powder liposomes manufactured by high shear mixing by the procedure described in Example 1 and as measured by the procedure described in Example 3.

FIG. 6 is a TEM (Transmission electron microscope)-negative stain images of Vitamin C tablets formulated with Vitamin C (PureWay-C®) fine powder liposomes manufactured by high shear mixing by the procedure described in Example 1 and as measured by the procedure described in Example 2.

FIG. 7 is a Cryo-TEM (Transmission electron microscope) images of Vitamin C tablets formulated with Vitamin C (PureWay-C®) fine powder liposomes manufactured by high shear mixing by the procedure described in Example 1 and as measured by the procedure described in Example 3.

FIG. 8 is a TEM (Transmission electron microscope)-negative stain images of Vitamin C gummy formulated with Vitamin C (PureWay-C®) fine powder liposomes manufactured by high shear mixing by the procedure described in Example 1 and as measured by the procedure described in Example 2.

FIG. 9 is a Cryo-TEM (Transmission electron microscope) images of Vitamin C gummy formulated with Vitamin C (PureWay-C®) fine powder liposomes manufactured by high shear mixing by the procedure described in Example 1 and as measured by the procedure described in Example 3.

FIG. 10 is a graph of concentration of vitamin C in 786-0 human epithelial cells following administration of the Vitamin C (PureWay-C®) fine powder liposomes manufactured by high shear mixing by the procedure described in Example 1; or non-liposomal vitamin C, as measured 10-120 minutes by the procedure described in Example 4.

FIG. 11 is a graph showing the percentage of wound healing area in 786-0 human epithelial cells following administration of the Vitamin C (PureWay-C®) fine powder liposomes manufactured by high shear mixing by the procedure described in Example 1; or non-liposomal vitamin C; or a control, as measured 0-48 hours by the procedure described in Example 5.

FIG. 12 is a graph showing the percentage of cells with neurites area in PC 12 human epithelial cells following administration of the Vitamin C (PureWay-C®) fine powder liposomes manufactured by high shear mixing by the procedure described in Example 1; or non-liposomal vitamin C; or a control, as measured 0-48 hours by the procedure described in Example 6.

FIG. 13 is a graph of the concentration of vitamin C in H9 human T-cells as measured 15-120 minutes by the procedure described in Example 7 following administration of the liposomal vitamin C (PureWay-C®) preparation of Example 1, or non-liposomal vitamin C.

FIG. 14 is a graph of the percentage inhibition of 1,1-diphenyl-2-picryl hydrazyl (DPPH) reduction as measured by the procedure described in Example 8 following administration of 1, 2.5, 5, 10, or 20 g/ml of the liposomal vitamin C (PureWay-C®) preparation of Example 1.

FIG. 15 is a graph of the percentage of cells exhibiting neurite outgrowth over 24 hours following administration of vehicle (-) or 0.5 M of the liposomal vitamin C (PureWay-C®) preparation of Example 1, or non-liposomal vitamin C, or a control, as measured by the procedure described in Example 9.

FIG. 16 is a graph showing the percentage of fibroblasts adhered to fibronectin substrates following administration of vehicle (-) or 50 M of the liposomal vitamin C (PureWay-C®) preparation of Example 1, or non-liposomal vitamin C, or as a control as measured by the procedure described in Example 10.

FIG. 17 is a graph of the plasma C-reactive protein levels in humans before and after supplementation over 24 hours following administration of the liposomal vitamin C (PureWay-C®) preparation of Example 1, or non-liposomal vitamin C, as measured by the procedure described in Example 11.

FIG. 18 is a graph of the plasma oxidized low density lipoprotein (oxLDL) levels in humans before and after supplementation over 24 hours following administration of the liposomal vitamin C (PureWay-C®) preparation of Example 1, or non-liposomal vitamin C, as measured by the procedure described in Example 11.

FIG. 19 is a graph of the concentration of serum vitamin C in human at various times prior (0 hours) and post supplementation as measured 1, 2, 4, 6 and 24 hours by the procedure described in Example 11, following administration of the liposomal vitamin C (PureWay-C®) preparation of Example 1, or non-liposomal vitamin C.

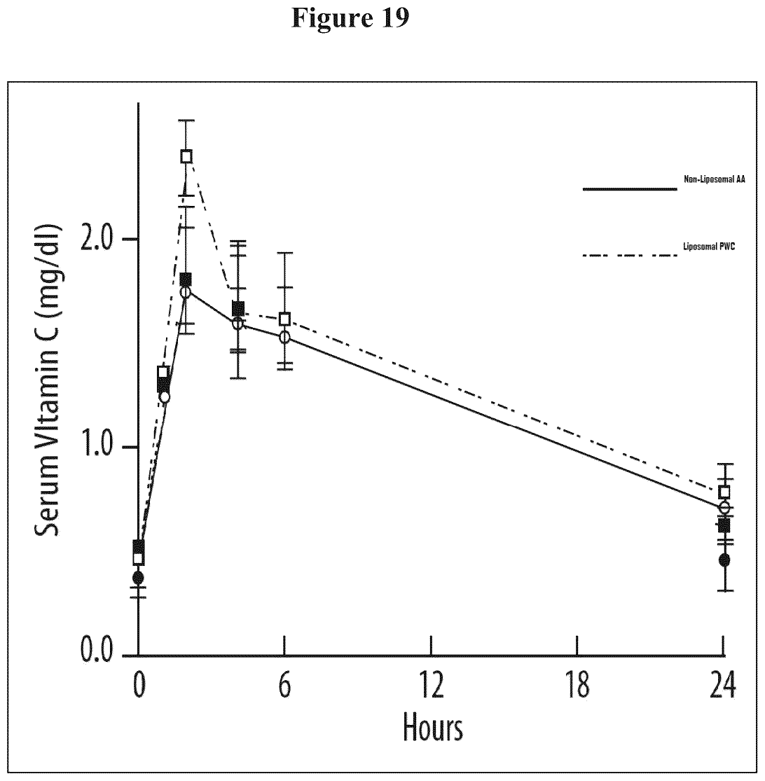

Like reference numerals refer to like parts throughout the several views of the drawings.

### DETAILED DESCRIPTION OF THE INVENTION

Before describing the present invention in detail, it is to be understood that unless otherwise indicated this invention is not limited to specific manufacturing methods, formulation components, dosage regimens pharmaceutical preparations, delivery systems or the like, as such may vary. In advance of the foregoing, the term “about” can be defined as what one skilled in the art would understand “about” to mean, and the term “about” includes a 5% tolerance on both lower and upper bounds, if applicable.

The invention now will be described more fully hereinafter with reference to the accompanying drawings in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

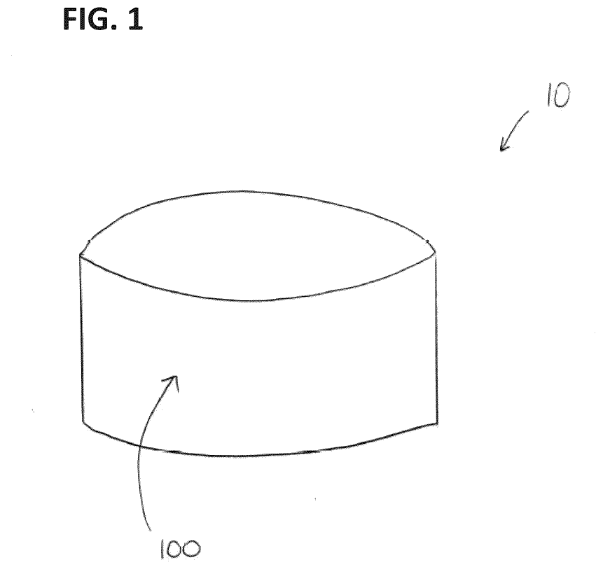

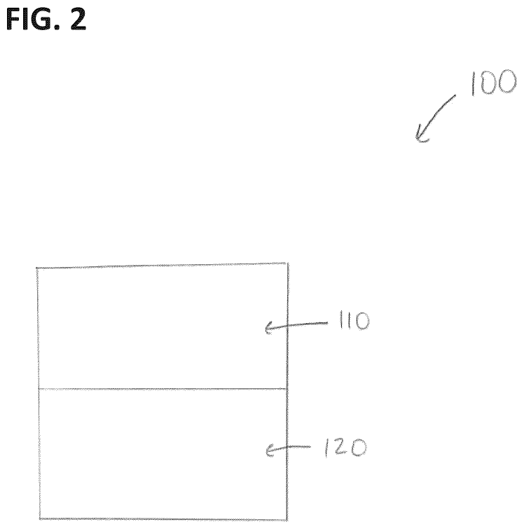

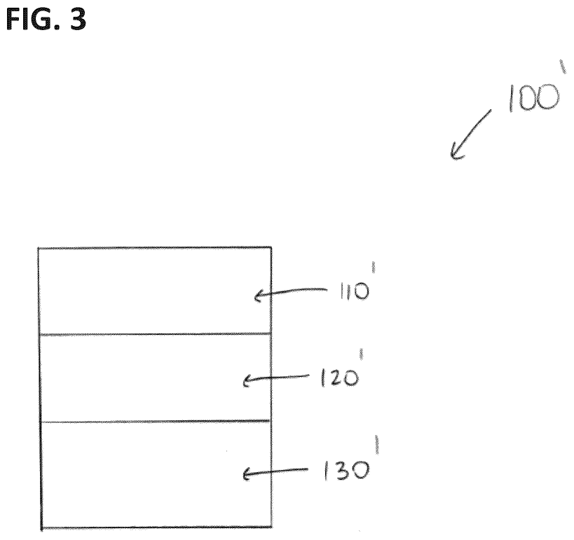

Turning now descriptively to the figures, FIGS. 1 and 2 illustrate one embodiment of an inventive liposome vitamin C preparation which enhances absorption of liposome vitamin C into cells and prolongs the retention of liposome vitamin C within the blood plasma and tissue of mammals, such as humans. FIG. 3, on the other hand, illustrates another embodiment of the inventive liposome vitamin C preparation.

FIGS. 1 and 2 show that the liposome vitamin C preparation 10 is primarily comprised of a homogenous high shear mixture 100, which itself is comprised of a vitamin C component 110 and an amphipathic and amphiphilic molecule component 120. In one embodiment of the present invention, illustrated in FIG. 3, the homogenous high shear mixture 100′ is primarily comprised of a vitamin C component 110′, an amphipathic and amphiphilic molecule component 120′, and a bioflavonoid component 130′.

In more specific terms, the liposome vitamin C preparation 10—preferably formulated as an oral dosage form, such as a tablet, capsule, softgel, or gummy—is comprised of a homogenous high shear mixture 100 which is further comprised of a vitamin C component 110 and an amphipathic and amphiphilic molecule component 120. The vitamin C component 110 of the homogenous high shear mixture 100 comprises at least about 74% by weight of the total weight of the preparation 10. In one embodiment of the present invention, the vitamin C component comprises about 0.01 g to 2.0 g of vitamin C. The amphipathic and amphiphilic molecule component 120, on the other hand, comprises at least about 0.1% by weight of the total weight of the preparation 10. The amphipathic and amphiphilic molecule component 120 further comprises at least one of the following: (i) at least one saturated straight C₁₄-C₂₄ fatty acid; (ii) at least one unsaturated ω-3 C₁₆-C₂₄ fatty acid; (iii) at least one unsaturated ω-6 C₁₈-C₂₂ fatty acid; and (iv) at least one unsaturated ω-7 C₁₆-C₂₀ fatty acid; and (v) at least one unsaturated ω-9 C₁₈-C₂₄ fatty acid; and (vi) at least one phospholipid at least one glycerophospholipid and at least one sphingophospholipid. According to one embodiment of the present invention, the homogenous high shear mixture comprises a weight ratio of vitamin C to amphipathic and amphiphilic molecules ranging from about 1000:1 to about 10:1. According to another embodiment, the weight ratio ranges from about 100:1 to about 6:1 to about 1:1 to about 1:3.

In one embodiment of the present invention, the homogenous high shear mixture may comprise a vitamin C component, an amphipathic and amphiphilic molecule component, and a bioflavonoid component. In such an embodiment, the bioflavonoid component comprises at least about 0.1% by weight of the total weight of the preparation. By way of non-limiting example, the homogenous high shear mixture may comprise at least about 74% by weight of the vitamin C component, at least about 0.1% to 65% by weight of the amphipathic and amphiphilic molecule component, and at least about 0.1% to 5% by weight of the bioflavonoid component. By way of another non-limiting example, the homogenous high shear mixture may comprise at least about 70% to 35% by weight of the vitamin C component, at least about 1% to 61% by weight of the amphipathic and amphiphilic molecule component, and at least about 0.1% to 5% by weight of the bioflavonoid component.

In another embodiment of the present invention, the homogenous high shear mixture may comprise at least about 0.01% by weight of one of at least one unsaturated ω-9 C₁₈-C₂₄ fatty acid, and at least about 0.1% by weight of one of at least one amphipathic and amphiphilic molecule component, based upon 100% total weight of the preparation.

By way of non-limiting example, the homogenous high shear mixture may further comprise a combination of at least one of the following: (i) at least about 0.01% by weight of at least one saturated straight C₁₄-C₂₄ fatty acid; (ii) at least about 0.01% by weight of at least one unsaturated ω-3 C₁₆-C₂₄ fatty acid; (iii) at least about 0.01% by weight of at least one ω-6 C₁₈-C₂₂ fatty acid; (iv) and at least about 0.01% by weight of at least one ω-7 C₁₆-C₂₀ fatty acid; (v) and at least about 0.01% by weight of at least one ω-9 C₁₈-C₂₄ fatty acid; (vi) and at least about 0.1% by weight of at least one amphipathic and amphiphilic molecule component, all of the foregoing based upon 100% total weight of the preparation.

In yet another embodiment of the present invention, the amphipathic and amphiphilic molecule component may comprise:
about 0.2-10.0 units (by weight) myristic acid;
about 0.2-10.0 units (by weight) pentadecanoic acid;
about 10-70.0 units (by weight) palmitic acid;
about 0.2-10.0 units (by weight) palmitoleic acid;
about 0.2-10.0 units (by weight) margaric acid;
about 0.5-60.0 units (by weight) stearic acid;
about 0.5-50.0 units (by weight) vaccenic acid;
about 5-80.0 units (by weight) oleic acid;
about 1-200.0 units (by weight) linoleic acid;
about 5-60.0 units (by weight) alpha linolenic acid;
about 5-60.0 units (by weight) linolenic acid;
about 0.2-10.0 units (by weight) arachidic acid;
about 0.2-10.0 units (by weight) gondoic acid;
about 0.2-10.0 units (by weight) behenic acid;
about 0.2-10.0 units (by weight) lignoceric acid;
about 0.2-10.0 units (by weight) nervonic acid;
about 10-50.0 units (by weight) 2-Lysophosphatidylcholine;
about 10-50.0 units (by weight) Diphosphatidylglycerole;
about 0.2-10.0 units (by weight) Lyso-Phosphatific Acid;
about 0.2-10.0 units (by weight) 1-Lyso-Phosphatidylcholine;
about 0.2-10.0 units (by weight) Lyso-Phosphatidylethanolamine;
about 1-50.0 units (by weight) N-Acyl-Phosphatidylethanolamine;
about 1-50.0 units (by weight) Phosphatidic Acid;
about 10.0-200.0 units (by weight) Phosphatidylcholine;
about 10.0-150.0 units (by weight) Phosphatidylethanolamine;
about 1-50.0 units (by weight) Phosphatidylglycerole;
about 10.0-200.0 units (by weight) Phosphatidylinositol; and
about 1-50.0 units (by weight) Phosphatidylserine,
   all based upon 100% total weight of the amphipathic and amphiphilic molecule component in the preparation.

By way of additional non-limiting example, the homogenous high shear mixture may comprise at least about 74% by weight of the vitamin C component and at least about 0.1% by weight of the amphipathic and amphiphilic molecule component, based upon 100% total weight of the preparation. More preferably, in such an example, the homogenous high shear mixture may comprise about 35% to about 71% by weight of the vitamin C component and from about 1% to about 65% by weight of the amphipathic and amphiphilic molecule component. According to yet another embodiment, the homogenous high shear mixture may comprise about 35% to about 70% by weight of the vitamin C component and from about 2% to about 65% (e.g., about 46%) by weight of the amphipathic and amphiphilic molecule component. According to one embodiment, the weight percentage of amphipathic and amphiphilic molecules in the amphipathic and amphiphilic molecule component preferably ranges from about 23.0% or 46.0% to 65.0% by weight, based upon the total weight of the preparation. By way of non-limiting example, the homogenous high shear mixture may comprise about 26% to about 1.8% by weight of amphipathic and amphiphilic molecules, and even more desirably, about 1.0% to about 61% by weight of amphipathic and amphiphilic molecules, based upon the total weight of the preparation. In yet another embodiment of the present invention, the amphipathic and amphiphilic molecule component is comprised of at least one of the following amphipathic and amphiphilic molecules at the following weight percentages:
about 0.02-1.0% (by weight) myristic acid;
about 0.02-1.0% (by weight) pentadecanoic acid;
about 1-7.0% (by weight) palmitic acid;
about 0.02-1.0% (by weight) palmitoleic acid;
about 0.02-1.0% (by weight) margaric acid;
about 0.05-6.0% (by weight) stearic acid;
about 0.05-5.0% (by weight) vaccenic acid;
about 0.5-8.0% (by weight) oleic acid;
about 1-20.0% (by weight) linoleic acid;
about 0.5-6.0% (by weight) alpha linolenic acid;
about 0.5-6.0% (by weight) linolenic acid;
about 0.02-1.0% (by weight) arachidic acid;
about 0.02-1.0% (by weight) gondoic acid;
about 0.02-1.0% (by weight) behenic acid;
about 0.02-1.0% (by weight) lignoceric acid;
about 0.02-1.0% (by weight) nervonic acid;
about 0.1-5.0% (by weight) 2-Lysophosphatidylcholine;
about 0.1-5.0% (by weight) Diphosphatidylglycerole;
about 0.02-1.0% (by weight) Lyso-Phosphatific Acid;
about 0.02-1.0% (by weight) 1-Lyso-Phosphatidylcholine;
about 0.02-1.0% (by weight) Lyso-Phosphatidylethanolamine;
about 0.1-5.0% (by weight) N-Acyl-Phosphatidylethanolamine;
about 0.1-5.0% (by weight) Phosphatidic Acid;
about 1.0-20.0% (by weight) Phosphatidylcholine;
about 1.0-15.0% (by weight) Phosphatidylethanolamine;
about 0.1-5.0% (by weight) Phosphatidylglycerole;
about 1.0-20.0% (by weight) Phosphatidylinositol; and
about 0.1-5.0% (by weight) Phosphatidylserine,
   all based upon 100% total weight of the amphipathic and amphiphilic molecule component in the preparation.

With regard to the amphipathic and amphiphilic molecule component 120 of the present invention, the amphipathic and amphiphilic molecule component 120 comprises amphipathic and amphiphilic molecules which, in one embodiment of the present invention, are extracted from natural waxes and natural oils for increased amphipathic and amphiphilic. The amphipathic and amphiphilic molecules comprised within the amphipathic and amphiphilic molecule component 120 are termed “absorption promoters” and are vital components of cellular membranes (and are involved in the communication among individual cells), are pivotal in the regulation and control of cellular function, and functionally enhance entry into cells and transport via the body's active transport and simple diffusion-dependent pathways. These molecules act as vitamin C carriers to increase the intestinal absorption and tissue distribution of vitamin C and enhance cellular uptake kinetics, which allows for vitamin C to enter cells quicker and in a more safe and effective manner. Amphipathic and amphiphilic molecules can shuttle between a number of different pathways, and the effects of increased amphipathic and amphiphilicity include a mechanism for enhancing the uptake, distribution, and release kinetics; favorably affecting the volume of distribution; and ease of interaction with cells in a non-toxic way. Furthermore, amphipathic and amphiphilic molecules enhance the elimination of electrophilic lipid peroxidation products (in the ascorbylation pathway) and have implications for the prevention of chronic inflammatory diseases and oxidative stress. Suitable sources of amphipathic and amphiphilic molecules may include, but are not limited to, sugar cane wax, rice bran wax, carnauba wax, candelilla wax, Japan wax, ouricury wax, bayberry wax, shellac wax, sunflower wax, orange wax, beeswax, rice brand oil, sunflower lecithin, sunflower oil, canola oil, flax seed oil, wheat germ oil, MCT oil, and hemp oil. According to one embodiment of the present invention, the set of sources of amphipathic and amphiphilic molecules comprises rice bran wax, carnauba wax, candelilla wax, beeswax, rice brand oil, sunflower oil, canola oil, and hemp oil. According to another embodiment of the present invention, the set of sources of amphipathic and amphiphilic molecules comprises rice bran wax, rice brand oil, sunflower lecithin and sunflower oil.

With regard to the amphipathic and amphiphilic molecules of the present invention which comprise the amphipathic and amphiphilic molecule component 120 comprises amphipathic and amphiphilic molecule that may include, but are not limited to: (i) at least one saturated straight C₁₄-C₂₄ fatty acid; (ii) at least one unsaturated ω-3 C₁₆-C₂₄ fatty acid; (iii) at least one unsaturated ω-6 C₁₈-C₂₂ fatty acid; and (iv) at least one unsaturated ω-7 C₁₆-C₂₀ fatty acid; and (v) at least one unsaturated ω-9 C₁₈-C₂₄ fatty acid; and (vi) at least one phospholipid at least one glycerophospholipid and at least one sphingophospholipid. To this point, the amphipathic and amphiphilic molecules from the natural waxes and natural oils tremendously affects the ability of the vitamin C to be absorbed into the body of the cellular compartments to increase their bioactivity and collective health. The amphipathic and amphiphilic molecules produce a superiorly absorbed form of vitamin C has enhanced delivery, availability, absorption kinetics, distribution, uptake, concentration, and utilization efficacy of essential vitamin C in the human body.

With regard to the bioflavonoid component 130′ of the present invention, the bioflavonoid component 130′ comprises bioflavonoids that may include, but are not limited to: rutin, naringin, hesperidin, neohesperidin, neohesperidin dihydrochalcone, naringenin, hersperitin, nomilin, didymin, narirutin and gallic acid. According to one embodiment of the present invention, the bioflavonoid component comprises hesperidin, gallic acid, and optionally other bioflavonoids. According to another embodiment of the present invention, the bioflavonoid component comprises hesperidin, didymin, narirutin, and optionally other bioflavonoids. By way of non-limiting example, the bioflavonoid component may, for purposes of protecting the vitamin C preparation from oxidizers and contributing to the preparation's antioxidant capabilities, comprise:
about 1000-10000 ppm hesperidin;
about 15-500 ppm didymin;
about 30-500 ppm narirutin; per gram of bioflavonoids

By way of additional non-limiting example, the bioflavonoid component may, for purposes of protecting the liposome vitamin C preparation from oxidizers and contributing to the preparation's antioxidant capabilities, comprise:
about 1000-10000 ppm hesperidin;
about 15-500 ppm didymin;
about 30-500 ppm narirutin; per gram of bioflavonoids

In some embodiments of the present invention, the preparations and/or formulations which may comprise controlled release formulations, sustained release implants, healthy beverages, foodstuffs, dietary supplements, topical compositions, and a wide variety of food applications themselves may comprise a combination of active ingredients that can be administered to both humans and animals.

In other embodiments of the present invention, the liposome vitamin C preparation can be administered to a human subject(s) to (i) promote a healthy nervous system; (ii) prevent or decrease the risk of developing a neurodegenerative disease; (iii) enhance NGF-mediated neurite outgrowth; (iv) promote wound healing; (v) enhance fibroblast adhesion to and the interaction with the extracellular matrix; (vi) protect the immune system from xenobiotics; (vii) decrease the risk of developing an oxidative pathogenesis; and (viii) decrease the risk of developing cancer, cardiovascular diseases, respiratory infections, pulmonary diseases, lung infections, atherosclerosis, respiratory diseases, and other age-related diseases associated with cytotoxic, genotoxic, and proinflammatory mechanisms. In such embodiments, desired results may be achieved by first recognizing the efficacy of the present invention to accomplish one of the above-referenced purposes (e.g., to promote a healthy nervous system) and second, after such recognition, attending to the oral administration of an effective dose of the present invention to the appropriate human subject.

### Dosage Forms

The liposome vitamin C preparation is preferably in the form of an oral dosage form, such as beads, pellets, granules, capsules (soft or hard), sachets, tablets, powders, dispersible powders capable of effervescing upon addition of water, aqueous or oily suspensions, emulsions, syrups, elixirs, or lozenges. For example, the oral dosage form can be a chewable tablet or gum; oral liquid dosage form, such as a suspension in an aqueous or non-aqueous liquid solution; or an emulsion, which can be a soft drink, tea, milk, coffee, juice, sports drink, or water. In some embodiments, the liposome vitamin C preparation is incorporated into various products, such as nutritional supplements (including vitamins and multivitamins), foods (including health food products such as nutrition bars), and drinks (including fruit juices such as energy drinks).

The compositions, preparations, formulations, combinations, conjugations of the invention are suitable for oral administration and may be presented as solid dosage form units such as beads, pellets, granules, capsules, sachets, tablets, powders, powders capable of effervescing upon addition of water or suspensions and/or lozenges, each containing a predetermined amount of the active compound, although any pharmaceutically acceptable dosage form can be utilized, and also the compositions for oral administration may be presented as a liquid dosage forms such as pharmaceutically acceptable suspensions in aqueous liquors or non-aqueous liquids such as syrup an elixir, solution, or an emulsion, and also in soft drinks, tea, milk, coffee, juices, sports drinks and water. The compositions, preparations, formulations, combinations, conjugations of the invention are suitable also for topical use in conventional forms such as solutions, suspensions, lotions, emulsions, ointments, creams and gels.

Generally, the daily dosage of most embodiments of the liposome vitamin C preparation on a vitamin C weight basis can range from about 30 mg to 2 g. For instance, in one such embodiment, the daily dosage can be about 60 mg to 1 g or about 60 mg to about 500 mg. In a preferred embodiment, the daily dosage ranges from about 60 mg to about 500 mg (e.g., the daily dosage can be 400 mg). According to another embodiment, the daily dosage ranges from about 60 mg to about 200 mg (e.g., the daily dosage can be 60, 100, or 200 mg). In some embodiments, the daily dose can be achieved by administration of a single dosage form of the present invention or, alternatively, two or more such dosage forms. In a preferred embodiment, the daily dose is achieved by administration of only one or two dosage forms (e.g., once daily dosing or b.i.d.). Therefore, the present invention may include, but is not limited to, dosage forms containing 30, 60, 100, 200, 400, 500, or 1000 mg of the liposome vitamin C preparation (on a vitamin C weight basis).

Some embodiments of the liposome vitamin C preparation may also include one or more excipients or additives. Suitable excipients and additives may include, but are not limited to, additional antioxidants (e.g., phenolic compounds), inert diluents (such as lactose, sodium carbonate, calcium phosphate, and calcium carbonates), granulating and disintegrating agents (such as corn starch and algenic acid), binders (such as starch), lubricants (such as magnesium stearate, stearic acid and talc), preservatives (such as ethyl or propyl p-hydroxybenzoate), colorants, flavoring agents, release modifying agents, thickeners, and any combination of any of the foregoing. Suitable antioxidants may include, but are not limited to, bioflavonoids, flavonoids, flavonols, flavanones, flavones, flavonals, flavanolols, and flavanols.

Some embodiments of the present invention can also contain other components useful in formulating pharmaceutical preparations for administration to humans and animals, including swelling agents, surfactants, solvents, preservatives, inert diluents, stabilizers, granulating agents, buffers, lubricants, disintegrating agents, antioxidants, coating agents, binders and the like, all of which are standard in the pharmaceutical arts. Suitable inert solid diluents may comprise calcium carbonate, calcium phosphate and kaolin. Suitable diluents for soft capsules include, but are not limited to, water and oils such as peanut oil, liquid paraffin, corn oil, wheat germ oil, soybean oil, and olive oil.

In some embodiments of the present invention, aqueous suspensions or dispersions contain the liposome vitamin C preparation, for example, in fine powder form together with one or more suspension or dispersion (or wetting) agents. Suitable suspension agents may include, but are not limited to, sodium carboxymethylcellulose, methylcellulose, hydroxypropylmethylcellulose, sodium alginate, polyvinylpyrrolidone, gum tragacanth and gum acacia. Suitable dispersing or wetting agents may include, but are not limited to, lecithin, condensation products of an alkylene oxide with fatty acids, condensation products of ethylene oxide with long chain aliphatic alcohols, condensation products of ethylene oxide with partial esters derived from fatty acids and a hexitol such as polyoxyethylene sorbitol monooleate, or condensation products of ethylene oxide with partial esters derived from fatty acids and hexitol anhydrides.

Dispersible powders and granules suitable for preparation of an aqueous suspension by the addition of water contain the liposome vitamin C preparation, for example, together with a dispersing agent, wetting agent, or suspending agent. Suitable dispersing agents, wetting agents, and suspending agents include those mentioned above.

In some embodiments, oily suspensions may be formulated by suspending the liposome vitamin C preparation in an oil, such as a vegetable oil or a mineral oil. The oily suspensions may also contain a thickening agent such as carnauba wax, candelilla wax, rice bran wax, beeswax, hard paraffin, or cetyl alcohol.

The liposome vitamin C preparation may, in some embodiments, be in the form of an oil-in-water emulsion. The oily phase may be a vegetable-based oil or a mineral-based oil. Suitable emulsifying agents may include, but are not limited to, naturally occurring gums (such as acacia and tragacanth gum), naturally occurring phosphatides (such as soybean, lecithin, esters, and partial esters derived from fatty acids and hexitol anhydrides) and condensation products of partial esters with ethylene oxide (such as polyoxyethylene sorbitan monooleate).

Syrups and elixirs may be formulated with sweetening agents such as glycerol, propylene glycol, sorbitol, aspartame, or sucrose, and may also contain a demulcent, preservative, flavoring, or coloring agent.

In other embodiments, the liposome vitamin C preparation may be in a form suitable for administration by inhalation (e.g., as a finely divided powder or a liquid aerosol), or for parenteral administration (e.g., as a sterile aqueous or oily solution for intravenous, subcutaneous, intramuscular dosing or as a suppository for rectal dosing). Administration of the vitamin C preparation by these nonoral routes is advantageous, as administration by these methods avoids gastrointestinal side effects, which may accompany high doses of vitamin C released in the stomach.

In some embodiments, the liposome vitamin C preparation can be delivered topically, for example, to protect the skin from free radicals, promote wound healing (for instance, for healing cuts, abrasions, sun damage (e.g., sun burn), wrinkles, and scars), and/or reduce inflammation. The liposome vitamin C preparation of the invention is advantageous compared to what is currently available, because the invention provides superior penetration of vitamin C through the skin than vitamin C alone can accomplish. Transdermal delivery of the liposome vitamin C preparation permits systemic delivery of the liposome vitamin C while avoiding gastrointestinal side effects. In some embodiments, the topical formulation containing the liposome vitamin C preparation can be in the form of a solution, suspension, lotion, emulsion, ointment, cream, or gel. According to one embodiment of the present invention, the topical formulation is a cream or lotion. The formulation may include additional active ingredients. These formulations may be prepared by methods known in the art, and typically include a topically acceptable vehicle. One embodiment is a topical formulation containing about 0.5 to about 25% by weight of the liposome vitamin C preparation of the present invention, based upon 100% total weight of the topical formulation. For instance, the topical formulation can contain 0.5-2%, 1-2%, 1-5%, 110%, 5-15%, 5-20%, or 10-20% by weight of the liposome vitamin C preparation.

In one embodiment of the present invention, the liposome vitamin C preparation is used to coat a medical device that is then positioned to a desired target location within the body, whereupon the vitamin C preparation elutes from the medical device. In such an embodiment, the coating preferably includes a therapeutically effective amount of the liposome vitamin C preparation. In another embodiment, the medical device is positioned so that the liposome vitamin C preparation is released in a therapeutically effective amount to a targeted site, such as a diseased or injured tissue or organ. The device can be introduced temporarily or permanently into a mammal (e.g., a human) for the prophylaxis or therapy of a medical condition, or to augment the immune system. The device can be introduced subcutaneously, percutaneously, or surgically. The medical device can be selected from stents, synthetic grafts, artificial heart valves, artificial hearts, and fixtures to connect the prosthetic organ to the vasculature, venous valves, abdominal aortic aneurysm grafts, inferior venal caval filters, catheters including permanent drug infusing catheters, embolic coils, embolic materials used in vascular embolization mesh repair materials, a Dracon vascular particle orthopedic metallic plates, rods, screws, and vascular sutures.

The liposome vitamin C preparation may be formulated to provide immediate release or controlled release (e.g., sustained release) of the liposome vitamin C preparation, for example, to provide effective doses of liposome vitamin C over extended periods of time to prolong the biological activity and beneficial biochemical functions of liposome vitamin C. One embodiment of the invention is a controlled release dosage form (such as a solid dosage form) containing about 0.01 g to 2.0 g of vitamin C, about 1 mg to 100 mg of amphipathic and amphiphilic molecules, and about 1 mg to 500 mg of bioflavonoids. For example, the controlled release dosage form may release about 10 to about 35% by weight of the total liposome vitamin C preparation within about 2 hours in an in vitro dissolution test, and about 40 to about 70% by weight of the total vitamin C preparation within about 8 hours. According to another embodiment, the controlled release dosage form may release about 50% by weight of the total liposome vitamin C preparation within about 2 hours in an in vitro dissolution test, and more than 90% by weight of the total liposome vitamin C preparation within about 6 or 8 hours. Any type of controlled release system known in the art can be used. The in vitro dissolution test is conducted using the Basket Method (Apparatus 1) with 900 ml 0.1M HCl as the medium run at 100 RPM at a temperature of 37° C. The samples are filtered through Whatman filter paper #1 and the amount of liposome vitamin C is calculated based on the equivalence to standard dicholorophenol-indophenol solutions. Solid controlled release dosage forms (e.g., tablets) can be formulated (e.g., coated) so as to prolong the release of the liposome vitamin C preparation into the gastrointestinal tract, or to prevent the release of the liposome vitamin C preparation in the stomach in order to prevent or attenuate the gastrointestinal side effects which can accompany high doses of liposome vitamin C released in the stomach. For example, the liposome vitamin C preparation can be enteric coated so as to prevent significant release of the preparation in the stomach. Controlled release of the liposome vitamin C preparation can prolong therapeutic and/or immunoprotective systemic concentrations of vitamin C in a person.

One embodiment of the invention is a three-layer controlled release dosage form (e.g., a tablet) where each layer contains a liposome vitamin C preparation of the invention. The liposome vitamin C preparation of each layer can be the same or different. At least one of the layers provides controlled release of the liposome vitamin C preparation. For example, the dosage form can include (i) a first layer, (ii) a second layer, and (iii) an outer layer surrounding the first and second layers, where the first layer and outer layer provide controlled release of the liposome vitamin C preparation(s) and the second layer provides immediate release of the liposome vitamin C preparation. According to one variation of this embodiment, the outer layer releases substantially all (>90%) of the liposome vitamin C preparation in a controlled manner within 60, desirably 30, and even more desirably 20 minutes, as determined by the aforementioned in vitro dissolution test. The second layer provides immediate release of the liposome vitamin C preparation contained therein. Finally, the first layer releases the liposome vitamin C preparation contained therein in a controlled manner over at least 6 hours (e.g., substantially of the liposome vitamin C preparation may be released within 6-10 hours or 6-8 hours), as determined by the aforementioned in vitro dissolution test.

Transdermal patch devices can also provide controlled administration (e.g., continuous or other sustained administration) of the liposome vitamin C preparation. Methods for preparing controlled release transdermal formulations are known in the art. For example, the transdermal device may contain an impermeable backing layer which defines the outer surface of the device and a permeable skin attaching membrane, such as an adhesive layer, sealed to the outer layer in such a way as to create a reservoir between them wherein the therapeutic agent is placed (e.g., a bandage or patch (including a time released patch)).

Other suitable controlled release systems may include, but are not limited to, long-term sustained implants, aqueous or oily suspensions, emulsions, syrups, elixirs, or lozenges, chewable tablet or gum, foods, beverages, osmotic systems, and dissolution system (e.g., effervescent oral dosage form).

The liposome vitamin C preparation of the present invention is preferably administered orally to a mammal (e.g., a human), but it can also be administered by other routes of administration, such as intravenously or subcutaneously.

### Preparation of Formulation

The liposome vitamin C preparation of the present invention may be prepared by methods well known in the art, such as high shear mixing the vitamin C, amphipathic and amphiphilic molecules, optionally bioflavonoids, and any desired excipients. The following examples illustrate the invention without limitation.

### Example 1

### Preparation of the Liposome Vitamin C Preparation:

A jacketed high shear mixer was charged with dry powder of 50 kg of vitamin C, 75 kg of the amphipathic and amphiphilic prepared above and 5 kg of bioflavonoids. The mixer was then turned on (agitation is initiated—plows) to create a homogenous high shear mixture of dry powder. The highspeed shearing devices (choppers) were initiated for 1 minute. Hot water was then pumped through the jacket of the mixer to heat the mixture to 80° C. with continuous mixing (plows only) for 15 minutes for complete liposome encapsulation. The liposome encapsulated mixture was cooled by running chilled water (10° C.) through the jacket under continuous mixing for 1 hour until a free-flowing powder was formed. The powder was discharged into a double polyethylene-lined container and then passed through a comminuting mill running at approximately 2500 rpm equipped with a 0.15 mm screen. The milled powder was collected 3 into appropriately labeled, double polyethylene-lined drums and reconciled.

TABLE 1
Formulation of One Embodiment of the Present Invention

| Ingredients | Amount | |
|---|---|---|
| Vitamin C | 35-74% | |
| Amphipathic and Amphiphilic | 23-65% | |
| Molecules | | |
| myristic acid | 20-1000 | mg/g |
| pentadecanoic acid | 20-1000 | mg/g |
| palmitic acid | 1000-7000 | mg/g |
| palmitoleic acid | 20-1000 | mg/g |
| margaric acid | 20-1000 | mg/g |
| stearic acid | 50-6000 | mg/g |
| vaccenic acid | 50-5000 | mg/g |
| oleic acid | 500-8000 | mg/g |
| linoleic acid | 1000-20000 | mg/g |
| alpha linolenic acid | 500-6000 | mg/g |
| linolenic acid | 500-6000 | mg/g |
| arachidic acid | 20-1000 | mg/g |
| gondoic acid | 20-1000 | mg/g |
| behenic acid | 20-1000 | mg/g |
| lignoceric acid | 20-1000 | mg/g |
| nervonic acid | 20-1000 | mg/g |
| 2-Lysophosphatidylcholine | 100-5000 | mg/g |
| Diphosphatidylglycerole | 100-5000 | mg/g |
| Lyso-Phosphatific Acid | 20-1000 | mg/g |
| 1-Lyso-Phosphatidylcholine | 20-1000 | mg/g |
| Lyso-Phosphatidylethanolamine | 20-1000 | mg/g |
| N-Acyl-Phosphatidylethanolamine | 100-5000 | mg/g |
| Phosphatidic Acid | 100-5000 | mg/g |
| Phosphatidylcholine | 100-20000 | mg/g |
| Phosphatidylethanolamine | 1000-15000 | mg/g |
| Phosphatidylglycerole | 100-5000 | mg/g |
| Phosphatidylinositol | 1000-20000 | mg/g |
| Phosphatidylserine | 100-5000 | mg/g |
| Bioflavonoids (optional) | 0.1-5% | |
| hesperidin | 1000-10000 | ppm |
| didymin | 15-500 | ppm |
| narirutin | 30-500 | ppm |

*-mg/g = mg of component per g of total amphipathic and amphiphilic molecules

### Example 2

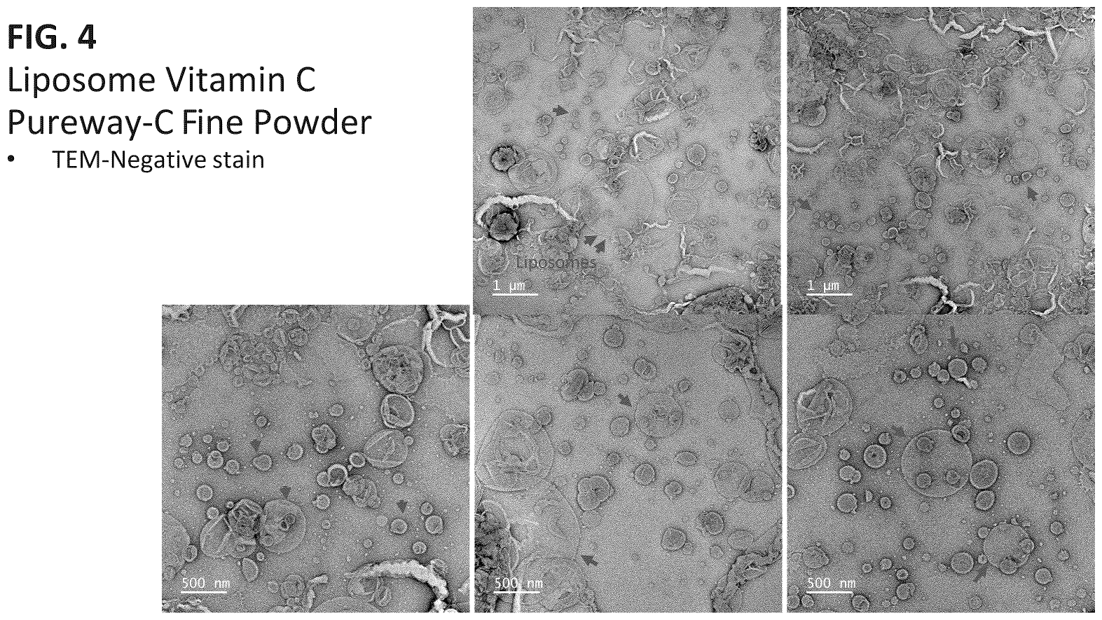

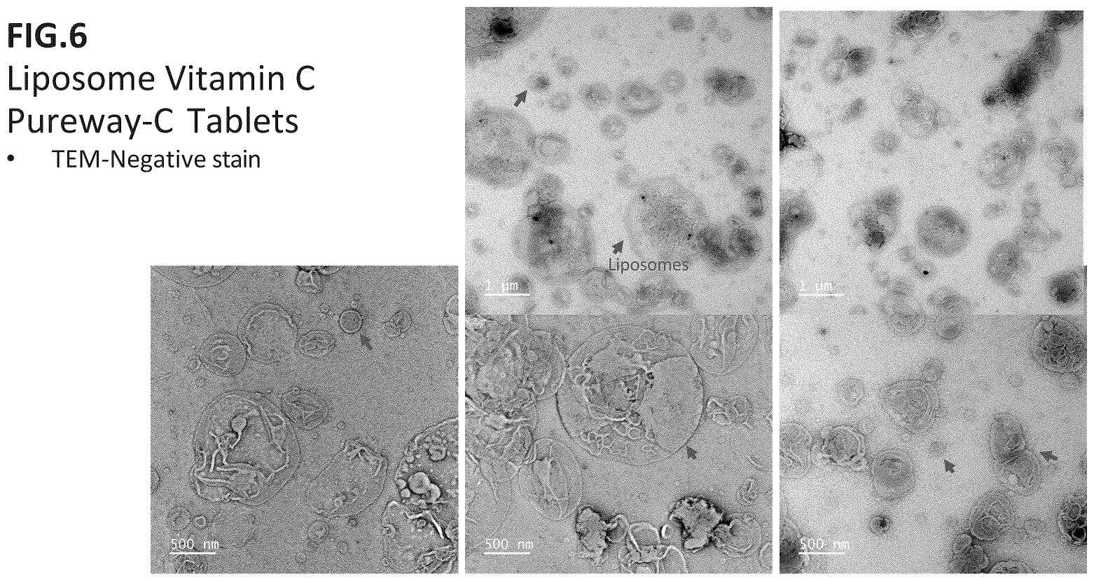

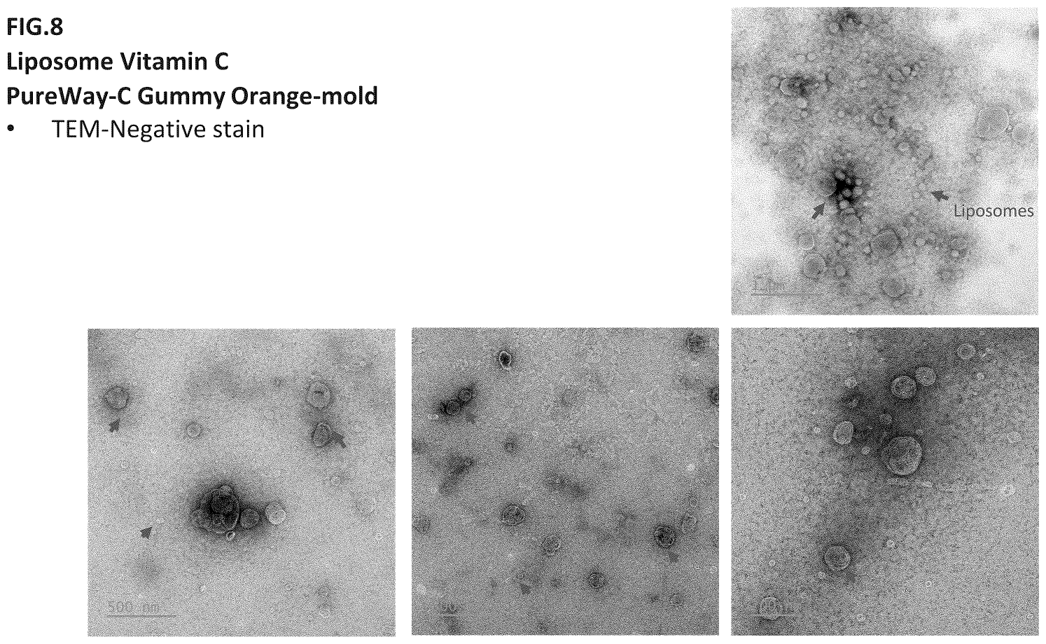

The TEM (Transmission electron microscope)-negative stain images of Vitamin C (PureWay-C®) fine powder liposomes was determined for the formulation of Example 1. 10 μL of the vitamin C (PureWay-C®) fine powder liposomes was placed onto a carbon coated 300 mesh copper grid that had been recently treated with plasma-discharge to make the carbon surface hydrophilic. After 60 seconds the excess specimen was removed by blotting and 10 μL of 1% phosphotungstic acid (PTA) in water, pH 7.2, was applied to the grid to contrast the specimen. The PTA was blotted off immediately. The grids were then viewed and images captured. Results are shown in FIGS. 4, 6, and 8.

### Example 3

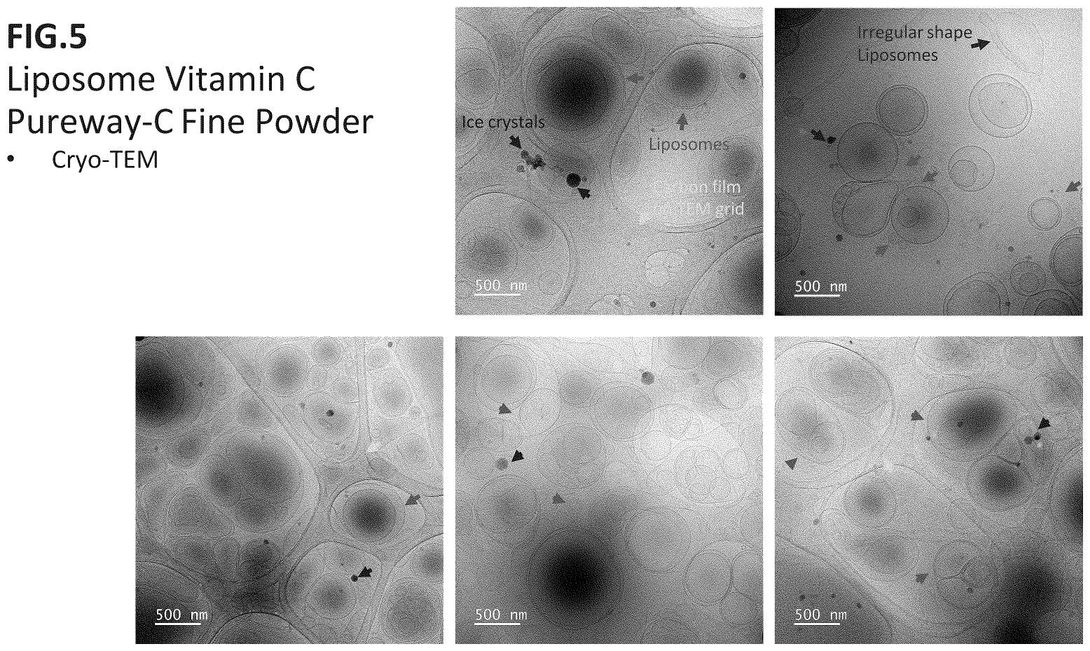

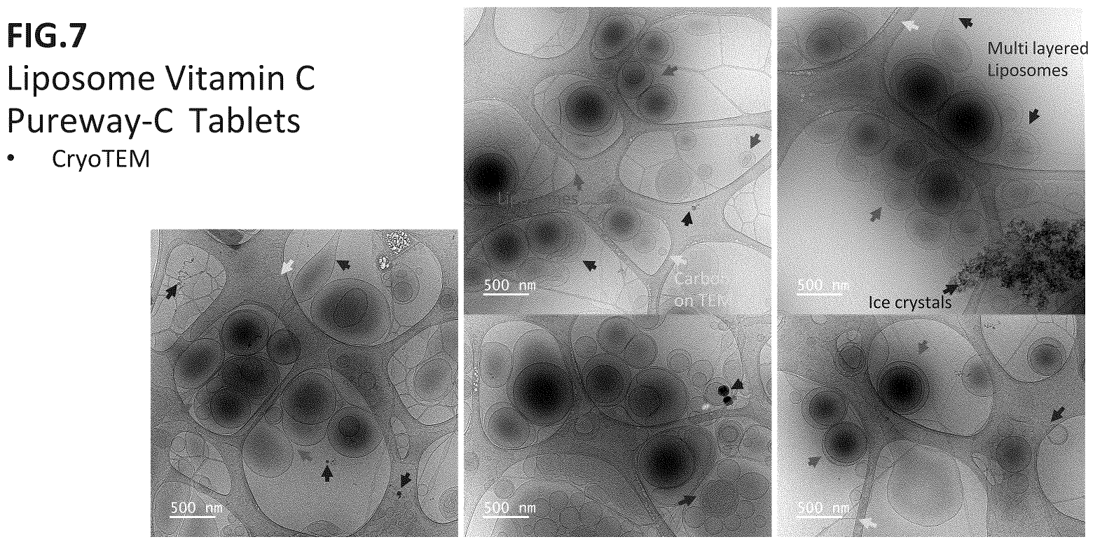

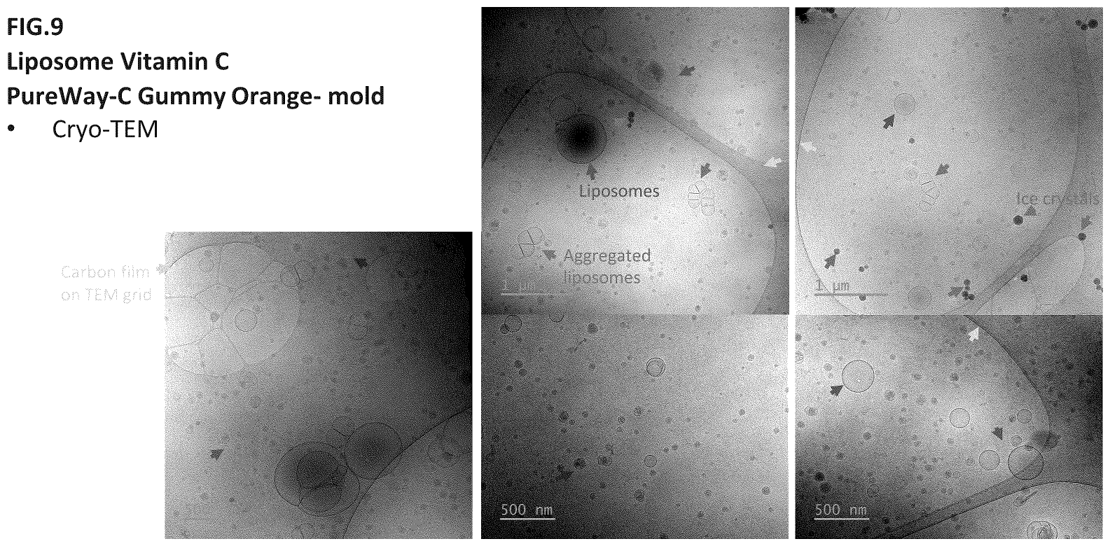

The Cryo-TEM (Transmission electron microscope) images of Vitamin C (PureWay-C®) fine powder liposome was determined for the formulation of Example 1. A 10 mg/ml solution (1:20 dilution of the stock solution) was deemed suitable for analysis. The liposomes in this sample seemed uniformly distributed. Both unilamellar and multilamellar liposomes were observed. Many of the observed small liposomes in the sample were multivesicular. Mainly round liposomes, but irregularly shaped liposomes were also commonly observed. The size range of liposomes found in our analysis was >100 nm. Approximately 29% of unilamellar, 58% multilamellar and 13% of multivesicular liposomes were observed (n=84 liposomes). Results are shown in FIGS. 5, 7, and 9.

### Example 4

The rate of absorption in human epithelia cells was determined for the formulation of Vitamin C (PureWay-C®) fine powder liposome (Example 1) and nonliposomal vitamin C.

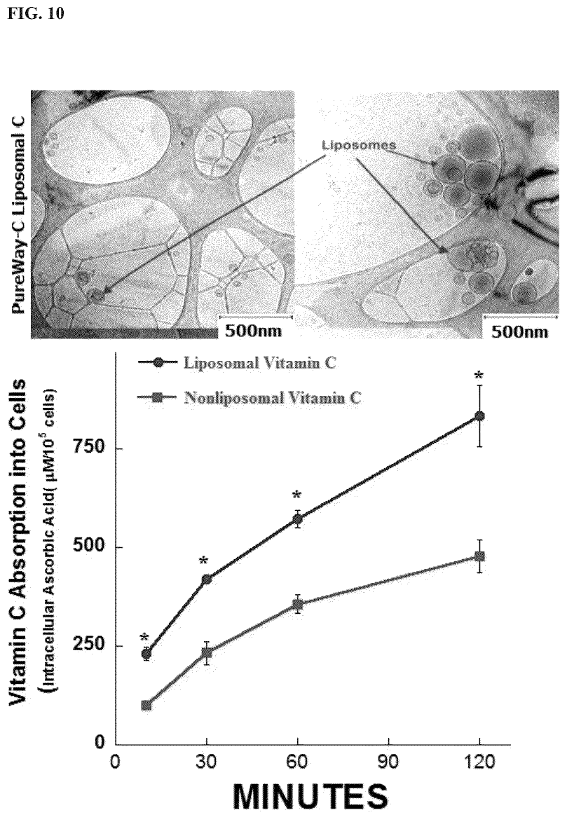

Liposomal vitamin C (PureWay-C®) is found in relatively unilamellar, multilamellar and multivesicular liposomes and better absorbed into human 786-0 epithelial cells. Top panel (FIG. 10): TEM of Liposomal vitamin C (PureWay-C®) showing liposomes in the 100-250 nm range (top panel-FIG. 10). The graph compares PureWay-C® liposomal C and nonliposomal vitamin C absorption into human epithelial cells. Human epithelial cells were seeded at 105 cells/well in 0.5 ml of serum free DMEM in wells of a 24 well tissue cluster plate. The cells were starved for 24 hours and then treated with 5 mM of liposomal vitamin C (blue line with circles) or nonliposomal vitamin C (red line with squares). The cells were incubated with the ascorbic acid for 10, 30, 60 and 120 minutes in triplicate after which the unabsorbed ascorbic acid was removed by aspiration and rinsing PBS, pH 7.2. The PBS was removed and the cells were lysed by three cycles of freeze-thawing for 10 minutes per cycle. The ascorbic acid EnzyChrom™ was then run on the cell lysate in the wells of the 24 well plate. When the chromagen was added the reaction was transferred to a 96 well plate and the OD was measured at 570 nm and the concentration of ascorbic acid was determined by comparison to a standard curve. The absorbed vitamin C is express as mM/105 cells. An ANOVA analysis of the data showed that the PureWay-C® liposomal C demonstrated statistically significant better absorption that the nonliposomal C at all time points tested at 95% confidence (*).

The liposomal vitamin C (PureWay-C®) showed better absorption, having more rapidly absorbed form of vitamin C and higher cellular levels of the same (233%) higher as compared to nonliposomal vitamin C as early as 10 minutes after treatment of the cells and at all time pointes tested. Results are shown in FIG. 10.

### Example 5

The percentage of wound area in human epithelia cells was determined for the formulation of Vitamin C (PureWay-C®) fine powder liposome (Example 1) and nonliposomal vitamin C.

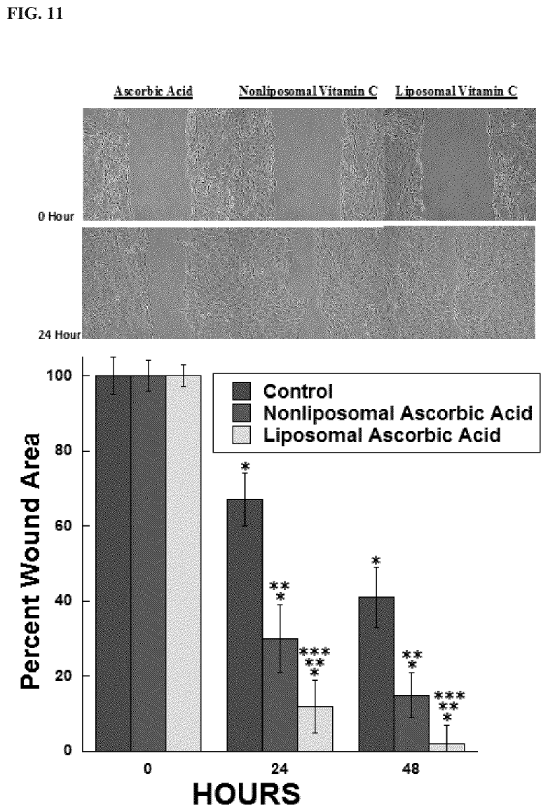

Top panel (FIG. 11): human kidney epithelial cells (786-0) were grown to 100% confluence to produce a monolayer. A wound in the monolayer was produced using a sterile 1000 μl micropipette tip and the cells were then immediately treated with 5 mM ascorbic acid, nonliposomal vitamin C, or PureWay-C® liposomal vitamin C (liposomal vitamin C) and incubated for 24 hours at 37° C. in a CO2 water-jacketed incubator. The monolayers were then photographed at the wound site immediately after treatment and at 24 and 48 hours. The above picture are representative images taken at time 0 (top row) and 24 hours (bottom row).

Graph: The size of the wound was determined at time 0, 24 hours and 48 hours by analyzing the images at 0, 24 and 24 hours with image. The wound area at time 0 is considered 100% and the percent wound area was determined for all treatments and time points. In an ANOVA analysis, all 24- and 48-hour treatments showed significant reduction in wound size from the previous day at 95% confidence (*). Further, the liposomal vitamin C (PureWay-C®) treatment lead to a significant increase of closure at 95% confidence (**). PureWay-C® liposomal vitamin C was significantly more effective than nonliposomal vitamin C at stimulating wound closure at 24 and 48 hours at 95% confidence (***). Results are shown in FIG. 11.

### Example 6

The percentage of cells with neurites in PC12 cells was determined for the formulation of Vitamin C (PureWay-C®) fine powder liposome (Example 1) and nonliposomal vitamin C.

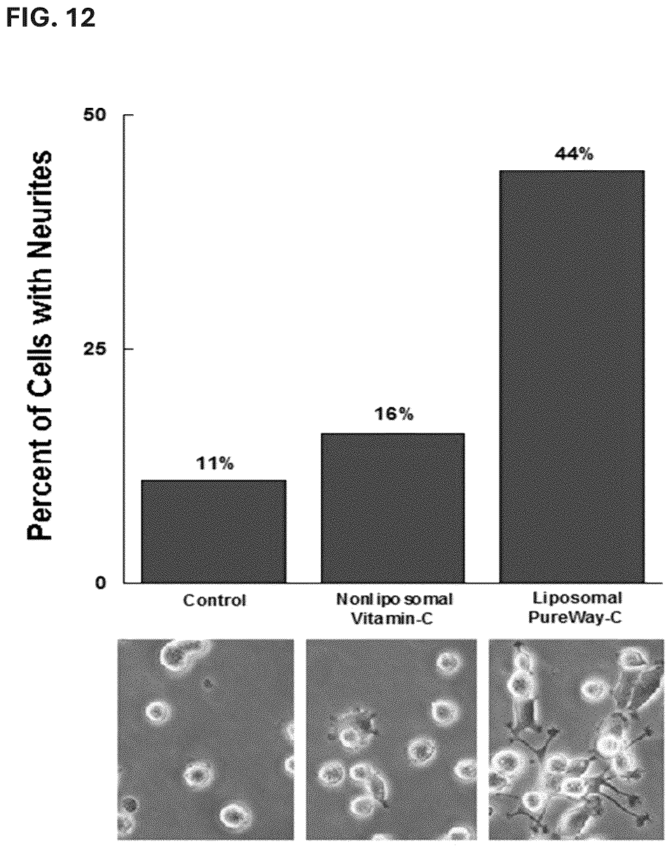

Top panel (FIG. 12): PC12 cells were treated with 100 ng/ml of Nerve Growth Factor (NGF) and incubated for a 24-hour period followed by treatment with either vehicle (or various 50 μM of liposomal vitamin C (PureWay-C®) or nonliposomal vitamin C. The formation of neurites was measured at hours 6. The results are shown in FIG. 12.

PC12 cells responded to NGF treatment by extending neurites. The liposomal vitamin C (PureWay-C®) preparation of Example 1 significantly enhanced the NGF-induced neurite outgrowth in 44% of the cells by the six hours. In fact, the liposomal vitamin C (PureWay-C®) preparation was the only formulation that resulted in a significant augmentation of NGF-induced neurite outgrowth, suggesting that this is the only formulation that would aid in protection against neurodegenerative diseases. In short, the neurites were forty-four-fold more efficient and the superiorly absorbed form of vitamin C promoted nerve regeneration more efficiently than nonliposomal vitamin C tested at 6 hours in time. Results are shown in FIG. 12.

### Example 7

The rate of absorption in H9 cells, a human T-cell line was determined for the formulation of Vitamin C (PureWay-C®) fine powder liposome (Example 1) and nonliposomal vitamin C

Liposomal vitamin C (PureWay-C®) is found in relatively unilamellar, multilamellar and multivesicular liposomes and better absorbed into H9 cells, a human T-cell line.

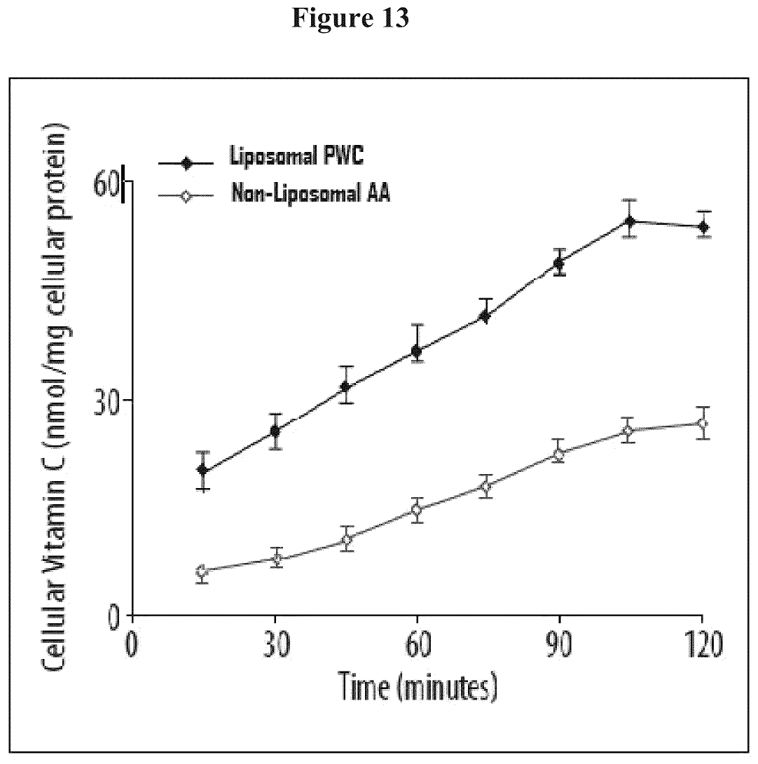

Cells from the human T-lymphoblastic H9 cell line were starved of vitamin C for 18 hours in serum-free media and subsequently suspended in 50 μM of liposomal vitamin C (PureWay-C®) preparation of Example 1, or nonliposomal vitamin C. At the times indicated in FIG. 13, cells were harvested and measured for vitamin C and protein content. The cellular vitamin C levels of the cells were measured.

Over a two-hour period, the level of vitamin C uptake from liposomal vitamin C (PureWay-C®) preparation of Example 1 was consistently higher than that observed with nonliposomal vitamin C (See FIG. 13). At fifteen minutes, cellular vitamin C levels ranged from 6±1.4 nmol/mg cellular protein with nonliposomal vitamin C ascorbic acid, to over quadruple that amount (25±2.4 nmol/mg protein) with the liposomal vitamin C (PureWay-C®) preparation of Example 1. The absorbed vitamin C levels rose significantly with time, peaking at approximately two hours with cellular levels ranging from 20 nmol/mg protein for nonliposomal vitamin C ascorbic acid and 59 nmol/mg protein for the liposomal vitamin C (PureWay-C®) preparation of Example 1.

In order for vitamin C to exert its beneficial effects, it must be taken up into the cell. To date, liposomal vitamin C (PureWay-C®) preparation of Example 1 exhibit the greatest amount of vitamin C uptake and retention as compared to nonliposomal vitamin C ascorbic acid. The result leads to human T-lymphocytes having a more rapidly absorbed form of liposomal vitamin C (PureWay-C®) preparation of Example 1 and higher cellular levels of the same (233% higher) as compared to nonliposomal vitamin C ascorbic acid tested at all time points. Results are shown in FIG. 13.

### Example 8

The antioxidant and free radical scavenging activities were determined for the liposomal vitamin C (PureWay-C®) preparation of Example 1.

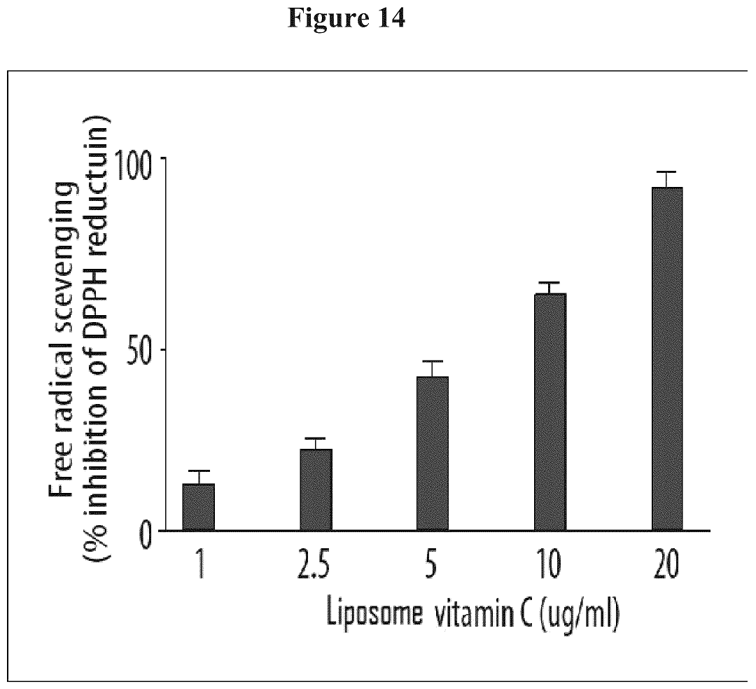

Briefly, 200 ml of a 1, 2.5, 5, 10, or 20 μg/ml solution of the vitamin C preparation of liposomal vitamin C (PureWay-C®) preparation of Example 1 was mixed with 50 μl of a 659 μM 1,1-diphenyl-2-picryl hydrazyl (DPPH) solution and incubated at 25° C. for 20 minutes. Free radical scavenging activity of the liposomal vitamin C (PureWay-C®) preparation of Example 1 was measured by the reduction of 1,1-diphenyl-2-picryl hydrazyl (DPPH) to 1,116 diphenyl-2-picryl hydrazine at an absorbance of 510 nm. The results are shown in FIG. 14.

The vitamin C preparation dose dependently scavenged DPPH free radicals. The liposomal vitamin C (PureWay-C®) preparation of Example 1 preparation demonstrated excellent scavenging ability by reducing the DPPH-induced free radical concentration by 98% at its maximum concentration.

Vitamin C is a chemical reducing agent for many intracellular and extracellular reactions such as oxidative DNA or protein damage, low-density lipoprotein oxidation, lipid peroxidation, oxidants, the formation of nitrosamines in gastric juice, extracellular oxidants from neutophils, and endothelium dependent vasodilation. The liposomal vitamin C (PureWay-C®) preparation of Example 1 of the present invention, which exhibits potent antioxidant and free radical scavenging effects in vitro, can serve as a good vitamin C preparation to prevent such damage thus contributing to the protection against cancer, cardiovascular diseases, respiratory infections, pulmonary diseases, lung infections, atherosclerosis, respiratory diseases, and other age-related diseases caused by cytotoxic, genotoxic, and proinflammatory mechanisms. Results are shown in FIG. 14.

### Example 9

The ability to promote neurite outgrowth was determined for the formulation of the liposomal Vitamin C (PureWay-C®) fine powder liposome (Example 1) and nonliposomal vitamin C.

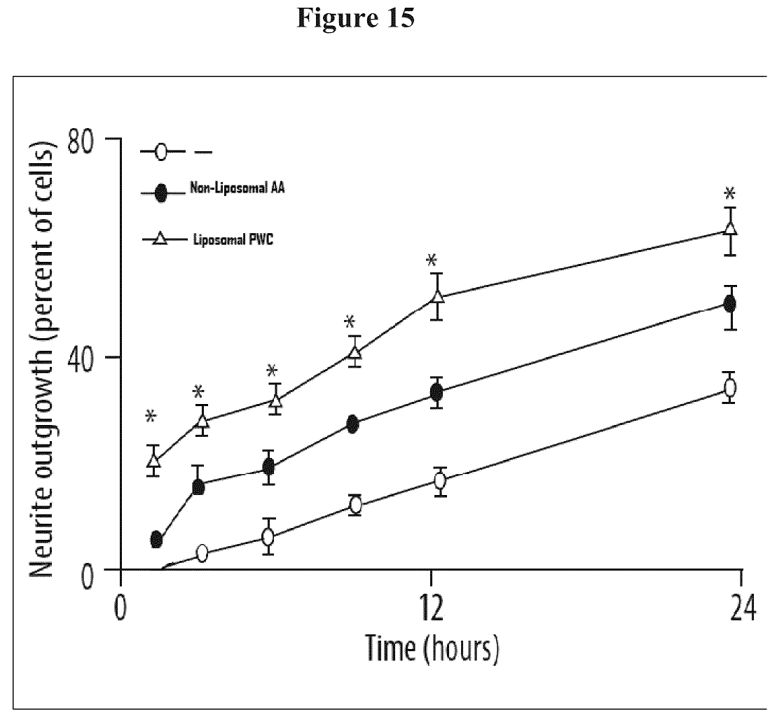

PC12 cells were treated with 100 ng/ml of Nerve Growth Factor (NGF) and incubated for a 24-hour period followed by treatment with either vehicle (-) or various 50 μM of liposomal vitamin C (PureWay-C®) preparation of Example 1, or nonliposomal vitamin C ascorbic acid. The formation of neurites was measured at hours 1, 3, 6, 9, 12, and 24. The results are shown in FIG. 15.

PC12 cells responded to NGF treatment by extending neurites. The liposomal vitamin C (PureWay-C®) preparation of Example 1 significantly enhanced the NGF-induced neurite outgrowth in 22% of the cells by the first hour. In fact, the liposomal vitamin C (PureWay-C®) preparation of Example 1 was the only formulation that resulted in a significant augmentation of NGF-induced neurite outgrowth, suggesting that this is the only formulation that would aid in protection against neurodegenerative diseases. In short, the neurites were twelve-fold more efficient and the superiorly absorbed form of vitamin C promoted nerve regeneration more efficiently than nonliposomal vitamin C ascorbic acid tested at all points in time. Results are shown in FIG. 15.

### Example 10

The ability to promote fibroblast adhesion to fibronectin was determined for the liposomal Vitamin C (PureWay-C®) fine powder liposome (Example 1) and nonliposomal vitamin C. NIH3T3 fibroblastoma cells were seeded onto fibronectin coated plates pretreated with either vehicle (-) or various 50 mM of liposomal vitamin C (PureWay-C®) preparation of Example 1, or nonliposomal vitamin C. The plates were incubated for 15 minutes at 37° C. The unattached cells were removed by aspiration and the attached cells were fixed, stained, and counted in triplicate.

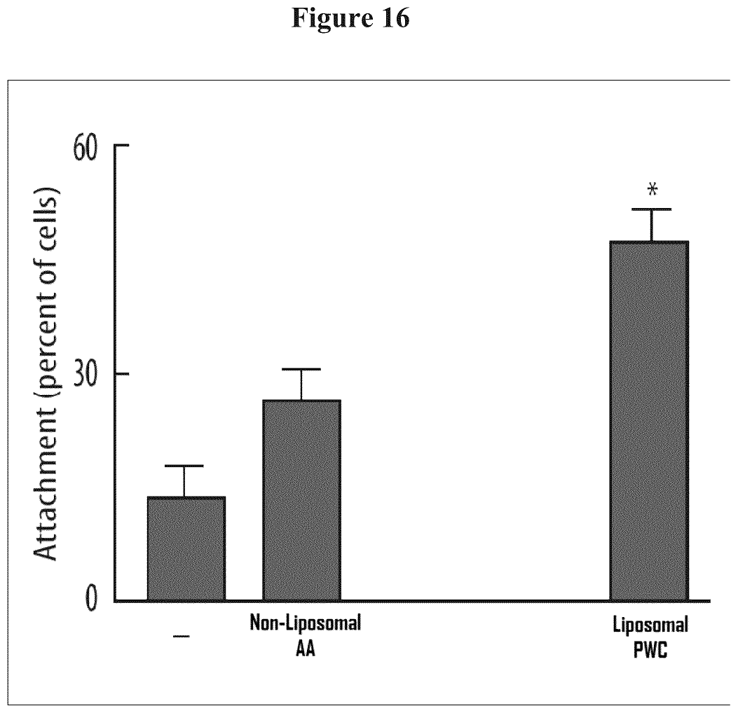

Results are shown in FIG. 16.

The liposomal vitamin C (PureWay-C®) preparation of Example 1 enhanced fibroblast adhesion to fibronectin by over six-fold. The liposomal vitamin C (PureWay-C®) preparation of Example 1 stimulated wound healing more efficiently than nonliposomal vitamin C ascorbic acid tested at all points in time. In addition to adhesion, fibroblast spreading on fibronectin is an important next step to migration and wound healing performance. Results are shown in FIG. 16.

### Example 11

The human serum vitamin C, plasma C-reactive protein, oxidized LDL, and urine uric and oxalate levels were determined for the liposomal Vitamin C (PureWay-C®) fine powder liposome (Example 1) and nonliposomal vitamin C.

Healthy volunteers maintained a low vitamin C diet for 14 days. Following an overnight fast, volunteers received a single oral dose of 1000 mg or either liposomal vitamin C (PureWay-C®) preparation of Example 1 or nonliposomal vitamin C ascorbic acid. Blood samples were collected immediately prior to the oral dose administration and at various time points post ingestion. Urine was collected over a 24-hour time period and saved for oxalate and uric acid assays. Serum vitamin C levels were measured by HPLC with coulometric electrochemical detection. Plasma C-reactive protein and oxidized LDL were measured by enzyme linked immunosorbent assay (ELISA) and urine uric acid and oxalate levels were measured by enzymatic methods.

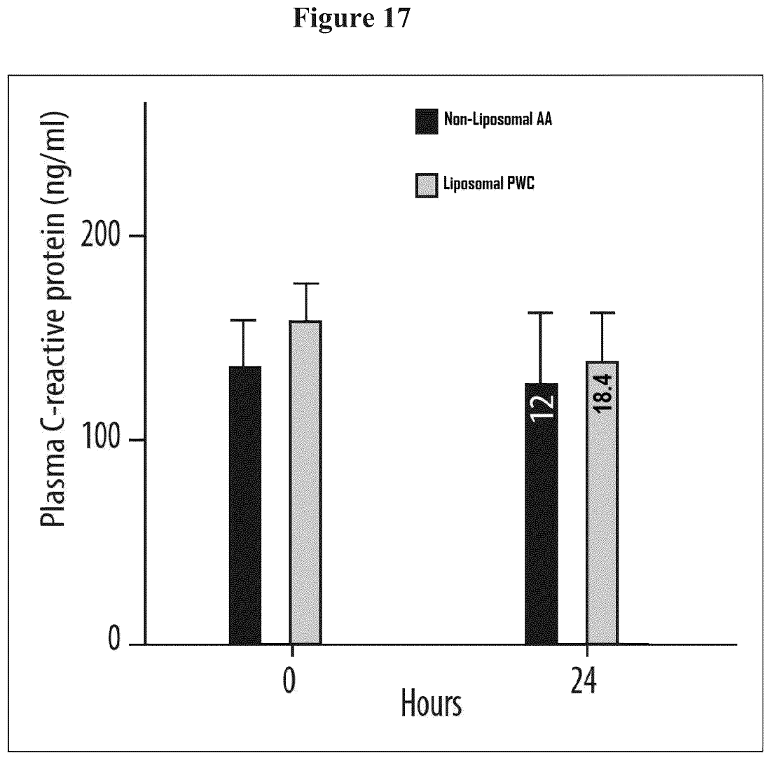

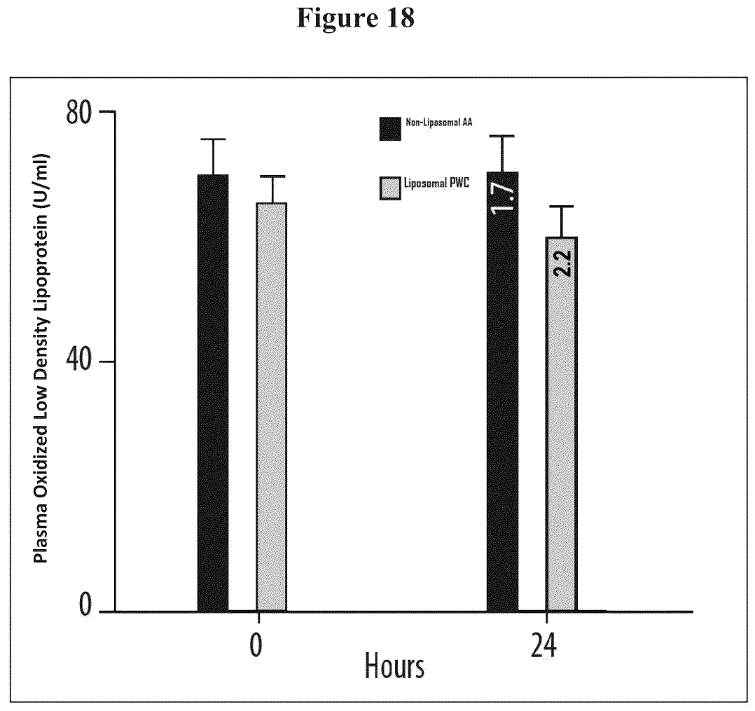

As can be seen in FIG. 17, and FIG. 18 the liposomal vitamin C (PureWay-C®) preparation of Example 1 best reduces the plasma levels of C-reactive protein (72% higher) and oxidized LDL (91% higher) in human clinical studies. As can be seen in FIG. 14, on the other hand, the liposomal vitamin C (PureWay-C®) preparation of Example 1 delivers effective free radical scavenging activity (21% higher) using the DPPH method.

The liposomal vitamin C (PureWay-C®) preparation of Example 1 is more rapidly absorbed and leads to higher serum vitamin C levels and greater reduction of plasma levels of inflammatory and oxidative stress markers than nonliposomal vitamin C ascorbic acid.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method for improving the status of a health condition comprising:
identifying a health condition, and
administering a liposome vitamin C preparation comprising a liposome vitamin C component, the liposome vitamin C component comprising at least about 35% by weight of the total weight of the liposome vitamin C preparation.

2. The method as recited in claim 1, wherein the liposome vitamin C preparation further comprises an amphipathic and amphiphilic molecule component, the amphipathic and amphiphilic molecule component comprising at least about 0.1% by weight of the total weight of the liposome vitamin C preparation.

3. The method as recited in claim 2, wherein the amphipathic and amphiphilic molecule component comprises at most about 65% by weight of the total weight of the liposome vitamin C preparation.

4. The method as recited in claim 2, wherein the amphipathic and amphiphilic molecule component comprises (i) at least one saturated straight C₁₄-C₂₄ fatty acid; (ii) at least one unsaturated ω-3 C₁₆-C₂₄ fatty acid; (iii) at least one unsaturated ω-6 C₁₈-C₂₂ fatty acid; (iv) at least one unsaturated ω-7 C₁₆-C₂₀ fatty acid; (v) at least one unsaturated ω-9 C₁₈-C₂₄ fatty acid; (vi) at least one phospholipid; (vii) at least one glycerophospholipid; and (viii) at least one sphingophospholipid.

5. The method as recited in claim 4, wherein the amphipathic and amphiphilic molecule component comprises (i) at least about 0.01% by weight of at least one saturated straight C₁₄-C₂₄ fatty acid; (ii) at least about 0.01% by weight of at least one unsaturated ω-3 C₁₆-C₂₄ fatty acid; (iii) at least about 0.01% by weight of at least one ω-6 C₁₈-C₂₂ fatty acid; (iv) at least about 0.01% by weight of at least one ω-7 C₁₆-C₂₀ fatty acid; and (v) at least about 0.01% by weight of at least one ω-9 C₁₈-C₂₄ fatty acid.

6. The method as recited in claim 4, wherein the amphipathic and amphiphilic molecule component comprises:
about 0.02-1.0% (by weight) myristic acid;
about 0.02-1.0% (by weight) pentadecanoic acid;
about 1-7.0% (by weight) palmitic acid;
about 0.02-1.0% (by weight) palmitoleic acid;
about 0.02-1.0% (by weight) margaric acid;
about 0.05-6.0% (by weight) stearic acid;
about 0.05-5.0% (by weight) vaccenic acid;
about 0.5-8.0% (by weight) oleic acid;
about 1-20.0% (by weight) linoleic acid;
about 0.5-6.0% (by weight) alpha linolenic acid;
about 0.5-6.0% (by weight) linolenic acid;
about 0.02-1.0% (by weight) arachidic acid;
about 0.02-1.0% (by weight) gondoic acid;
about 0.02-1.0% (by weight) behenic acid;
about 0.02-1.0% (by weight) lignoceric acid;
about 0.02-1.0% (by weight) nervonic acid;
about 0.1-5.0% (by weight) 2-Lysophosphatidylcholine;
about 0.1-5.0% (by weight) Diphosphatidylglycerole;
about 0.02-1.0% (by weight) Lyso-Phosphatific Acid;
about 0.02-1.0% (by weight) 1-Lyso-Phosphatidylcholine;
about 0.02-1.0% (by weight) Lyso-Phosphatidylethanolamine;
about 0.1-5.0% (by weight) N-Acyl-Phosphatidylethanolamine;
about 0.1-5.0% (by weight) Phosphatidic Acid;
about 1.0-20.0% (by weight) Phosphatidylcholine;
about 1.0-15.0% (by weight) Phosphatidylethanolamine;
about 0.1-5.0% (by weight) Phosphatidylglycerole;
about 1.0-20.0% (by weight) Phosphatidylinositol; and
about 0.1-5.0% (by weight) Phosphatidylserine.

7. The method as recited in claim 1, wherein the liposome vitamin C preparation further comprises a bioflavonoid component, the bioflavonoid component comprising at least about 0.1% by weight of the total weight of the liposome vitamin C preparation.

8. The method as recited in claim 7, wherein the bioflavonoid component comprises at most about 5% by weight of the total weight of the liposome vitamin C preparation.

9. The method as recited in claim 1, wherein the health condition comprises nervous system health.

10. The method as recited in claim 1, wherein the health condition comprises a risk of developing a neurodegenerative disease.

11. The method as recited in claim 1, wherein the health condition comprises neurite outgrowth via NGF mediation.

12. The method as recited in claim 1, wherein the health condition comprises wound healing.

13. The method as recited in claim 1, wherein the health condition comprises fibroblast adhesion to and the interaction with human extracellular matrices.

14. The method as recited in claim 1, wherein the health condition comprises a protection offered by human immune systems against xenobiotics.

15. The method as recited in claim 1, wherein the health condition comprises a risk of developing an oxidative pathogenesis.

16. The method as recited in claim 1, wherein the health condition comprises a risk of developing cancer.

17. The method as recited in claim 1, wherein the health condition comprises a risk of developing cardiovascular diseases.

18. The method as recited in claim 1, wherein the health condition comprises a risk of developing respiratory infections.

19. The method as recited in claim 1, wherein the health condition comprises a risk of developing pulmonary diseases.

20. The method as recited in claim 1 wherein the health condition comprises a risk of developing lung infections.

21. The method as recited in claim 1, wherein the health condition comprises a risk of developing atherosclerosis.

22. The method as recited in claim 1, wherein the health condition comprises a risk of developing respiratory diseases.

23. The method as recited in claim 1, wherein the health condition comprises a risk of developing age-related diseases associated with cytotoxic mechanisms.

24. The method as recited in claim 1, wherein the health condition comprises a risk of developing age-related diseases associated with genotoxic mechanisms.

25. The method as recited in claim 1, wherein the health condition comprises a risk of developing age-related diseases associated with proinflammatory mechanisms.
